(19) United States
(12) Patent Application Publication
(10) Pub. No.: US 2025/0297168 A1
(43) Pub. Date: Sep. 25, 2025

(54) PROCESS AND APPARATUS FOR SEPARATING PARAFFINS

(71) Applicant: UOP LLC, Des Plaines, IL (US)

(72) Inventor: Scott Lyle Nauert, Chicago, IL (US)

(21) Appl. No.: 19/050,792

(22) Filed: Feb. 11, 2025

Related U.S. Application Data
(60) Provisional application No. 63/569,044, filed on Mar. 22, 2024.

(51) Int. Cl. C10G 7/00

(52) U.S. Cl. CPC C10G 7/00

(57) ABSTRACT

A process for separating paraffins is disclosed. The process comprises separating a feed stream into a first vapor stream and a first liquid stream. The first vapor stream is fed to a first distillation column at a first inlet. A liquid side stream is taken from the first distillation column. The liquid side stream is separated into a second vapor stream and a second liquid stream. The second vapor stream and said first liquid stream are fractionated in the first distillation column. The liquid side stream is taken from an outlet located above an inlet for passing the first liquid stream in the first distillation column.

### FIELD

The field is the separation of light paraffins. The field may particularly relate to separation of paraffins from a naphtha to light paraffinic reactor effluent stream.

### BACKGROUND

Light olefin production is vital to the production of sufficient plastics to meet worldwide demand. Dehydrogenation is a process in which light paraffins such as ethane and propane can be dehydrogenated to make ethylene and propylene, respectively, typically in the presence of a catalyst. Dehydrogenation can be achieved in either the presence of an oxidant such as oxygen or in the absence of an oxidant. Non-oxidative dehydrogenation is an endothermic reaction which requires external heat to drive the reaction to completion. Propane dehydrogenation (PDH) is a widely practiced example of non-oxidative dehydrogenation to produce propylene from propane. Ethane oxidative dehydrogenation is a newer oxidative process for converting ethane to ethylene which can be conducted at lower temperatures with lower carbon oxide emissions than non-oxidative and thermal cracking processes.

Fluid catalytic cracking (FCC) is another endothermic process that can be tuned to produce substantial propylene. However, not every FCC unit is tuned to make substantial propylene. Also, high propylene FCC units do not recover much ethylene; less than 1% of global ethylene supply comes from FCC.

The great bulk of the ethylene consumed in the production of plastics and petrochemicals such as polyethylene is produced by the thermal cracking of hydrocarbons. Steam is usually mixed with the feed stream to a cracking furnace to reduce the hydrocarbon partial pressure and enhance olefin yield and to reduce the formation and deposition of carbonaceous material in the cracking reactors. The process is therefore often referred to as steam cracking or pyrolysis.

Paraffins with a range of carbon numbers can be thermally cracked to produce olefins including ethane, propane, butanes, and naphtha. Ethane and naphtha are typical thermal cracking feeds due to their higher yield to light olefins than propane and butane feeds. Ethane feed is used in regions where light hydrocarbon gases are prevalent. In regions where gas is not abundant, naphtha feed is employed for steam cracking. Naphtha steam cracking has long set the price in the ethylene industry due to higher production cost versus ethane steam cracking. The world does not currently produce enough ethane to supply the growing demand for ethylene. Therefore, regions lacking ethane supply such as Asia and Europe rely mainly on naphtha steam cracking to supply ethylene. Naphtha steam cracking yields only about 30%-35% ethylene with the balance including both relatively high-value by-products comprising propylene, butadiene, and butene-1 and relatively low value by-products comprising pyoil, pygas, and fuel gas. Additional pressures on naphtha steam cracking including minimum production requirements and environmental concerns have led to the withholding of government approvals in certain regions such as China. The ethylene industry needs a more efficient, economical and environmentally friendly route to light olefins from naphtha feeds.

### BRIEF SUMMARY

A process for separating paraffins comprises separating a feed stream into a first vapor stream and a first liquid stream. The first vapor stream is fed to a first distillation column at a first inlet. A liquid side stream is taken from the first distillation column. The liquid side stream is separated into a second vapor stream and a second liquid stream. The second vapor stream and said first liquid stream are fractionated in the first distillation column. The liquid side stream is taken from an outlet located above an inlet for passing the first liquid stream to the first distillation column. By taking the liquid side stream from an outlet located above an inlet for passing the first liquid stream and passing the second vapor stream taken from the liquid side stream into the first distillation column, the present process prevents the backmixing of an ethane rich liquid with a C3+ hydrocarbon rich feed inside the first distillation column, thereby lowering the utility expense. The present process also provides an improved recovery of ethane product.

### BRIEF DESCRIPTION OF THE DRAWINGS

FIG. 1 is a schematic drawing of an embodiment of the process and apparatus of the present disclosure.

FIG. 2 is a schematic drawing of another embodiment of the process and apparatus of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

As used herein, the term “predominant” or “predominate” means greater than 50%, suitably greater than 75% and preferably greater than 90%.

The term “Cx” is to be understood to refer to molecules having the number of carbon atoms represented by the subscript “x”. Similarly, the term “Cx−” refers to molecules that contain less than or equal to x and preferably x and less carbon atoms. The term “Cx+” refers to molecules with more than or equal to x and preferably x and more carbon atoms.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottoms stream back to the bottom of the column. Feeds to the columns may be preheated. The top pressure is the pressure of the overhead vapor at the vapor outlet of the column. The bottom temperature is the liquid bottom outlet temperature. Overhead lines and bottoms lines refer to the net lines from the column downstream of any reflux or reboil to the column.

As used herein, the term “separator” means a vessel which has an inlet and at least an overhead vapor outlet and a bottoms liquid outlet and may also have an aqueous stream outlet from a boot. A flash drum is a type of separator which may be in downstream communication with a separator that may be operated at higher pressure.

### DETAILED DESCRIPTION

Naphtha and liquefied petroleum gas (LPG) feed stock comprising C3-C8 hydrocarbons is primarily charged to a “Naphtha to Ethane and Propane” (NEP) reactor to convert naphtha and LPG in the presence of hydrogen into desirable ethane and propane along with less desirable methane. The ethane and propane may be fed to an ethane steam cracker and propane dehydrogenation unit to make ethylene and propylene, respectively. To improve the purity of the ethane stream, a demethanizer column is used which separates light ends such as hydrogen and methane in an overhead product and C2+ hydrocarbons in a bottoms product. The C2+ bottoms product contains heavier molecules and is fed to a downstream deethanizer column to recover a purified ethane stream. Separation in the demethanizer column involves a backmixing step in which a heavy feed mixes with a separated ethane-rich liquid flowing down the column before sending the mixed stream to an upper reboiler for heat recovery. Back mixing the ethane-rich liquid from the column with heavier components in the heavy feed increases the temperature at which methane is stripped off in the upper reboiler which is disadvantageous for heat recovery. The backmixing due to mixing of the heavy feed with the ethane-rich liquid also leads to an entropy loss which should be avoided. This mixing step reverses some of the separation achieved by the column which must be re-separated in the downstream deethanizer column, resulting in an increased condenser and reboiler duty in the downstream deethanizer column.

The current process successfully prevents mixing the separated ethane-rich liquid with the heavy feed by one or more of the following: removing ethane rich liquid from the demethanizer column above the feed location, partially stripping methane from the ethane rich liquid in an upper reboiler, separating the reboiled ethane rich stream into a methane rich vapor stream that is returned to the column, and taking the further enriched ethane liquid as a product. The ethane rich liquid product and the demethanizer bottoms product can then be further separated in a downstream deethanizer column to produce an ethane rich product with applicable purity specification. An advantage of the current process is that the upper reboiler operates at lower temperature which is advantageous for heat recovery. A second advantage of the current process is that it produces ethane that meets the applicable purity specification with lower utilities expense compared with comparable alternatives.

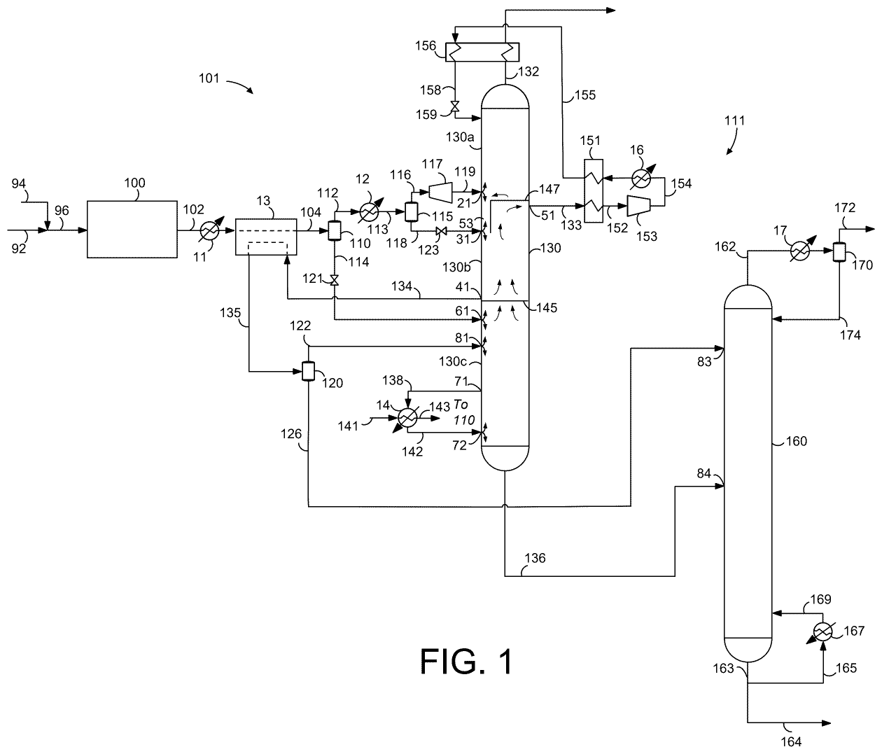

Turning to FIG. 1, an embodiment of a process and an apparatus for separating paraffins 101 is disclosed. The process comprises a naphtha to ethane and propane (NEP) unit 100, and a separation unit 111. The NEP unit may comprise a NEP reactor and a splitter column. A naphtha stream in line 92 may be combined with a hydrogen stream in line 94 to provide a charge stream in line 96. The charge stream in line 96 may be heated and charged to a naphtha to ethane and propane (NEP) reactor 100 to be contacted with an NEP catalyst. The naphtha stream in line 92 may comprise C4 to C12 hydrocarbons preferably having a T10 between about −10° C. and about 60° C. and a T90 between about 70 and about 180° C. The naphtha feed stream may comprise normal paraffins, iso-paraffins, naphthenes, and aromatics. The naphtha stream may be heated to a reaction temperature of about 300° C. to about 600° C., suitably between about 325° C. and about 550° C., and preferably between about 350° C. and about 525° C. Weight space velocity should be between about 0.3 to about 20 hr⁻¹, suitably between about 0.5 and about 10 hr⁻¹ and preferably between about 1 to about 4 hr⁻¹. A total pressure should be about 0.1 to about 3 MPa (abs), suitably no less than about 1 MPa (abs) and preferably from about 1.5 to about 2.5 MPa (abs). At these conditions, C2-C4 paraffinic yield is consistently in an excess of 80 wt %, while methane yield is less than about 16 wt %. The hydrogen-to-hydrocarbon ratio should be about 0.3 to about 15 and preferably about 0.5 to about 5. In a further embodiment, the hydrogen-to-hydrocarbon molar ratio may typically be no more than 5, suitably be no more than 3 and preferably be no more than 2.

The NEP catalyst for converting naphtha to ethane and propane may contain a molecular sieve comprising large or medium pores, that is, comprising 10 or 12 member rings, respectively. Examples of suitable molecular sieves include MFI, MEL, MFI/MEL intergrowth, MTW, TUN, UZM-39, IMF, UZM-44, UZM-54, MWW, UZM-37, UZM-8, UZM-8HS. Examples of suitable molecular sieves further include FER, AHT, AEL (SAPO-11), AFO (SAPO-41), MRE, MFS, EUO-1, TON (ZSM-22), MTT (ZSM-23) and UZM-53. Additional molecular sieves with larger pores include FAU, EMT, FAU/EMT intergrowth, UZM-14, MOR, BEA, UZM-50, MTW, ZSM-12. Additional examples include MSE and UZM-35.

MFI is a suitable NEP catalyst. It will be appreciated that ZSM-5 is an MFI-type aluminosilicate zeolite belonging to the pentasil family of zeolites and having a chemical formula of NaₙA1ₙSi₉₆-nO₁₉₂·16H₂O (0<n<10). In various embodiments, the ZSM-5 zeolite may comprise a silica-to-alumina molar ratio of 20 to 1000, 20 to 800, 20 to 600, 20 to 400, 20 to 200 or 20 to 80. In various embodiments, the ZSM-5 zeolite may comprise a crystal size in the range of 10 to 600 nm, 20 to 500 nm, 30 to 450, 40 to 400 nm, or 50 to 300 nm.

The NEP catalyst may comprise a bound zeolite. The binder may comprise an oxide of aluminum, silicon, zinc, titanium, zirconium and mixtures thereof. The binder may comprise a phosphate in the binder or a phosphate of the forenamed oxide binder materials. Preferably, the binder is a silicon oxide. The MFI zeolite may be supported in a silicon oxide containing binder or an alumina containing binder such as aluminum phosphate.

MFI zeolite slurry may be first mixed with a binder in the form of colloidal suspension (sol) and gelling reagent and then dropped into hot oil to make spheres controlled to produce ⅛-inch to about 1/32-inch diameter calcined supports. Alternatively, the zeolite may be mixed with a silicon oxide containing binder and extruded to 1/32 to ¼-inch diameter extrudates. Extrudates may be washed with ammonia to remove sodium ions from the zeolite, dried and calcined to remove the organic structural directing agent (OSDA) from the synthesized zeolite. Optionally, the calcined support may be ammonium-ion exchanged using an ammonium nitrate solution to remove residual sodium ions and dried at about 110° C.

The NEP catalyst comprises a metal on the catalyst. The metal may comprise a transition metal. The metal may be a noble metal. In a further example, the metal may comprise platinum, palladium, iridium, rhenium, ruthenium and mixtures thereof. A modifier metal may also be included on the catalyst. The modifier metal may include tin, germanium, gallium, indium, thallium, zinc, silver and mixtures thereof. The modifier metal should be more concentrated on the binder than on the zeolite. About 0.01 to about 5 wt % of each the transition metal and the modifier metal may be on the catalyst.

Metal may be incorporated into the binder by evaporative impregnation. A solution of platinum such as tetraamine platinate nitrate or chloroplatinic acid may be contacted with the bound spherical or extrudate supports which have been calcined and ion-exchanged in a rotary evaporator, followed by drying and oxidation.

The NEP catalyst comprises a metal on the bound spherical or extrudate supports of the catalyst. Preferably, more of the metal is on the binder than on the zeolite. At least 60 wt %, suitably at least 70 wt %, preferably at least 80 wt % and most preferably at least 90 wt % of the metal is on the binder. The zeolite and/or the entire NEP catalyst is steamed oxidized to drive the metal off the zeolite. Steaming is preferably effected after the metal is added to the catalyst. The dried, impregnated spherical or extrudate supports may be steam oxidized in air for sufficient time to provide NEP catalysts. Steam oxidation in air at a temperature of about 500° C. to about 650° C. and about 5 mol % to about 30 mol % steam for about 1 to 3 hours may be suitable.

The NEP catalysts must be reduced to activate them for catalyzing the NEP reaction. For example, the catalyst may be reduced in flowing hydrogen at about 500 to about 550° C. for 3 hours before contacting feed.

After paraffin conversion, a reactor effluent stream comprising paraffins is discharged from the NEP reactor. The reactor effluent stream may be a light paraffinic stream. The C4-fraction of the reactor effluent stream may comprise at least about 40 wt % ethane or at least about 40 wt % propane or at least about 70 wt % and preferably at least about 80 wt % ethane and propane. The ethane to propane ratio can range from about 0.1 to about 5. The C4-fraction of the reactor effluent stream can have less than about 16 wt %, suitably less than about 14 wt %, preferably less than about 12 wt %, and more preferably less than about 10 wt % methane.

The reactor effluent stream may be cooled and fed to a splitter column to separate aromatics in a splitter heavy stream. The splitter heavy stream is taken from the splitter column in a bottoms line and may be rich in aromatics. In an embodiment, the splitter heavy stream may be recycled to the NEP reactor to absorb the exotherm.

A splitter light stream rich in paraffins is taken in an overhead line of the splitter column. A splitter light stream may be passed to the separation unit 111 to separate one or more paraffins from the splitter light stream. In an embodiment, the splitter light stream may be taken in splitter receiver overhead line as a splitter overhead vapor stream in line 102.

The overhead vapor stream in line 102 may comprise hydrogen, methane, ethane, and C3+ hydrocarbons. The overhead vapor stream in line 102 may be taken as a feed stream and passed to the downstream NEP separation unit 111. The NEP separation unit 111 may be a fractionation column or a series of fractionation columns and other separation units. In accordance with the present disclosure, the separation unit 111 may comprise a first distillation column 130 and a second distillation column 160. In an exemplary embodiment, the first distillation column 130 is a demethanizer column. In another exemplary embodiment, the second distillation column 160 is a deethanizer column.

The feed stream comprising hydrogen, methane, ethane, and C3+ hydrocarbons in line 102 may be cooled in a heat exchanger 11 to a first temperature. In an exemplary embodiment, the feed stream in line 102 may be cooled by heat exchange with a demethanizer overhead vapor stream in line 132 in the heat exchanger 11. The feed stream in line 102 may be further cooled in a heat exchanger 13 with a liquid side stream in line 134 taken from the demethanizer column 130. A cooled feed stream in line 104 may be discharged from the heat exchanger 13 and passed to a first separator 110 The feed stream in line 102 may be cooled to a temperature of about to about −10° C. (14° F.) to about −80° C. (−112° F.) in the heat exchanger 13 The first separator 110 separates the cooled feed stream in line 104 into a first vapor stream in line 112 and a first liquid stream in line 114. There is no restriction on how cooling the feed stream in line 102 to the first temperature is achieved. It is beneficial to utilize as much heat from the process as possible. To do this, it is beneficial to cool all or a portion of the feed stream against the demethanizer overhead product, the demethanizer bottom product, the demethanizer lower reboiler liquid, a downstream ethane product, or a combination of the four. Cooling the feed stream to the first temperature can also be supplied in part by an external refrigerant.

In an exemplary embodiment, the feed stream in line 104 may be split into a first feed stream and a second feed stream. The first feed stream may be cooled against a downstream ethane product stream and the demethanizer overhead product in a heat exchanger. The second feed stream may be cooled against a portion of the demethanizer bottom product in a heat exchanger, followed by cooling against a lower reboiler liquid in a sequential heat exchanger. The partially cooled first and second feed streams may be combined and optionally cooled against external refrigerant in a heat exchanger. The refrigerant can have any composition. Preferable options are propane, propylene, or a mixed refrigerant whose composition is matched to the combined partially cooled feed condensing curve. The combined partially cooled feed is then separated in the first separator 110 into the first vapor stream in line 112 and a first liquid stream in line 114.

The feed stream in line 102 may comprise non-condensable gases such as hydrogen. Other non-condensable gases include oxygen, carbon monoxide and nitrogen which are not applicable to the NEP context but which may be applicable to other separations processes for which the disclosed process and apparatus are beneficial. Other separations comprising non-condensable gases which may benefit by the disclosed process and apparatus include but are not limited to natural gas liquids separation and separation of light hydrocarbons from a methanol to olefins process. These non-condensable gases are primarily separated into the first vapor stream in line 112. The first vapor stream in line 112 may be cooled to a second temperature in a heat exchanger 12 to provide a cooled first vapor stream in line 113. In an embodiment, the first vapor stream in line 112 may be cooled to a temperature of about −50° C. (−58° F.) to about −100° C. (−148° F.) in the heat exchanger 12. In an exemplary embodiment, the first vapor stream in line 112 may be cooled to a temperature of about −70° C. (−94° F.) to about −90° C. (−130° F.) in the heat exchanger 12. The cooled first vapor stream in line 113 may be expanded across one or more pressure letdown devices to provide further cooling and optionally recover power then fed to one or more locations to the demethanizer column 130. In an exemplary embodiment, the cooled first vapor stream in line 113 may be separated in a second separator 115 to provide a third vapor stream in line 116 and a third liquid stream in line 118. The third vapor stream in line 116 and the third liquid stream in line 118 are fractionated in the demethanizer column 130. The third vapor stream in line 116 or an expanded vapor stream 119 may be passed to a first inlet 21 to the demethanizer column 130. The third liquid stream in line 118 may be passed to a fourth inlet 31 to the demethanizer column 130. In an embodiment, the third liquid stream in line 118 is passed to the demethanizer column 130 at the fourth inlet 31 which is at or below a first inlet 21 for the third vapor stream in line 116. In an aspect, the third vapor stream in line 116 may be expanded across one or more pressure letdown devices 117 to provide further cooling and optionally recover power providing an expanded third vapor stream in line 119 which is fed to the demethanizer column 130. In an embodiment, the pressure letdown device is an expansion valve. In an exemplary embodiment, the pressure letdown device is a turboexpander. In another aspect, the third liquid stream in line 118 may be expanded by a valve 123 or a power recovery turbine to provide additional refrigeration, and an expanded third liquid stream is passed to the demethanizer column 130. In an exemplary embodiment, the third vapor stream in line 116 may be expanded from a pressure of about 3344 kPa (gauge) (485 psig) to about 1379 kPa (gauge) (200 psig) in the expander 117. Expanding the third vapor stream in line 116 in the expander 117 may further cool the third vapor stream in line 116 to a temperature of about −70° C. (−103° F.) to about −110° C. (−166° F.). The non-condensable gases present in the cooled first vapor stream in line 113 and subsequently present in the third vapor stream in line 116 move upwardly in the demethanizer column 130 and the condensable hydrocarbons primarily move downwardly in the column 130. In an embodiment, the third vapor stream in line 119 and the liquid stream in line 118 may be fed to the column 130 at the same elevation.

There is no restriction on how cooling to the second temperature is achieved. It is beneficial to utilize as much heat from the process as possible. To do this, it is beneficial to cool all or a portion of the first vapor stream in line 112 against the demethanizer overhead product, the demethanizer upper reboiler liquid, or a combination of the two. Cooling to the second temperature can also be supplied in part by an external refrigerant. The external refrigerant can have any composition. Preferable options are ethane, ethylene, or a mixed refrigerant whose composition is matched to the combined partially cooled third feed stream condensing curve.

In an embodiment, the first vapor stream 112 is expanded in a turboexpander 117 to provide the expanded third vapor stream 119 without cooling in heat exchanger 12 or separation in separator 115. In an exemplary embodiment, the first liquid stream in line 114 may be expanded across a valve 121 or a power recovery turbine to provide additional refrigeration. The expanded third vapor stream in line 119 and the expanded third liquid stream after valve 123 may be fed to the demethanizer column 130 at the same inlet 21 or different inlets 21 and 31 and/or elevations. The first liquid stream in line 114 may be passed to a third inlet 61 of the demethanizer column 130.

The third vapor stream in line 116 and the third liquid stream in line 118 are fractionated in the demethanizer column 130. In accordance with the present disclosure, a liquid side stream is taken from a first outlet 41 of the demethanizer column 130 in line 134. In an embodiment, the first outlet 41 of the demethanizer column 130 is located below the first inlet 21 for the third vapor stream in line 116 and the fourth inlet 31 for the third liquid stream in line 118. The first outlet 41 is suitably located on the demethanizer column 130 for withdrawing the liquid side stream in line 134 and separating it before recycling a portion of the liquid side stream back to the demethanizer column 130. The first outlet 41 is suitably located for withdrawing the liquid side stream in line 134 to prevent an ethane rich liquid from mixing with heavier materials in the feed such as the first liquid stream in line 114 which makes the separation easier in the downstream decthanizer column 160. In an embodiment, the demethanizer column 130 comprises a liquid tray 145. The first outlet 41 is located near and above the tray 145. The liquid coming down from above the tray 145 gets accumulated on the tray 145. The liquid which accumulates on the tray 145 is taken out from the demethanizer column 130 in the liquid side stream in line 134 from the first outlet 41. In an exemplary embodiment, the tray 145 is a total liquid accumulation tray which collects all the liquid from above onto it so that none of the liquid goes below through openings in the tray 145. All the liquid above the tray 145 in the column 130 that descends to the tray 145 is taken from the total liquid accumulation tray 145 in the liquid side stream in line 134 and removed from the first outlet 41 from the demethanizer column 130. The pneumatics of the column surrounding the tray 145 allow vapor coming from below the tray to pass through openings in the tray as shown by the arrow in the FIG. 1 but liquid may not pass downwardly through the openings in the tray perhaps due to the upcoming vapor. The first outlet 41 of the liquid side stream for line 134 is located at an elevation above a third inlet 61 which feeds the first liquid stream in line 114 to the demethanizer column 130. This way, the ethane-rich liquid descending in the column above the tray 145 does not mix with heavier hydrocarbons entering the demethanizer column 130 in the incoming liquid feed from the third inlet 61.

In an embodiment, a vapor side stream is taken from a second outlet 51 of the demethanizer column 130 in a side line 133. A tray 147 is provided in the demethanizer column 130. The vapor side stream in line 133 is taken from the second outlet 51 located near and below the tray 147. The position of the tray 147 is such that the vapors rising from below the tray 147 as shown by the arrows do not mix with the vapor components entering in the column with the third vapor stream in line 119. The pneumatics of the column operate to direct upwardly rising gases from below tray 147 out the second outlet 51 and allows liquid to travel down the downcomer 53 from above the tray. This arrangement provides a benefit that the non-condensable gases in the feed stream in the third vapor stream in line 119 or the third liquid stream in line 118 do not mix with purified condensable gases such as methane and ethane in the vapor side stream in line 133 withdrawn from second outlet 51. This way, the vapor side stream 133 comprises primarily condensable gases such as methane and ethane suitable for condensing to provide reflux to the column. This in turn allows for a higher condenser temperature and lower condenser duty than attempting to condense a stream comprising non-condensable gases in the feed

The vapor side stream in line 133 is heated in a side heat exchanger 151 by heat exchange with a cooled vapor side stream in line 154 to provide a heated vapor side stream in line 152. The heated vapor side stream in line 152 may be compressed in a compressor 153. In an exemplary embodiment, the heated vapor side stream in line 152 may be compressed from a pressure of about 1378 kPa (gauge) (200 psig) to about 3930 kPa (gauge) (570 psig) in the compressor 153. A compressed vapor side stream in line 154 is cooled in a heat exchanger 16. The cooled and compressed vapor side stream in line 154 is heat exchanged with the vapor side stream in line 133 in the side heat exchanger 151 to provide cooled vapor side stream 155. The cooled vapor side stream is taken in line 155 is further cooled to condense at least a portion of the stream to provide a reflux stream in line 158. The reflux stream in line 158 is then passed to the overhead of the demethanizer column 130. The reflux stream in line 158 may be expanded by passing to a valve 159 and an expanded reflux stream is then passed to the overhead of the demethanizer column 130. In an aspect, the cooled vapor side stream in line 155 is heat exchanged with an overhead vapor stream in line 132 in an overhead heat exchanger 156 to cool the reflux stream in line 158. In an aspect, the reflux stream in line 158 may be expanded before it is passed to the overhead of the demethanizer column 130. The vapor side stream in line 133 is compressed, cooled in the heat exchanger 16, and cooled against the demethanizer overhead product stream in line 132 in the overhead heat exchanger 156 to produce a mostly liquid stream in line 158 which is fed to the top of the demethanizer column 130 to provide reflux in the upper section. The upper section of the demethanizer column 130 is enriched in non-condensable gases such as hydrogen. By taking the vapor side stream in line 133 below the first feed stream in the third vapor stream in line 119 at the first inlet 21, the vapor side stream in line 133 is enriched in methane which can be condensed to provide reflux at the top of the demethanizer column 130 thereby lowering utilities. The first inlet 21 and the third inlet 61 separate the demethanizer column 130 into three sections. An upper section 130a is located above the first inlet 21 for the third vapor stream in line 116, a middle section 130b between the first inlet 21 and the third inlet 61, and a lower section 130c below the third inlet 61 for the first liquid stream in line 114. The liquid side stream in line 134 is taken from the first outlet at the bottom of the middle section 130b. The vapor side stream is taken in line 133 from the second outlet 51 from a the top of the middle section 130b below the first inlet 21 for the third vapor stream in line 116. The temperature of the upper reboiler 13 and the pressure of the third separator 120 control the methane fraction dissolved in the ethane rich product in line 126. In an exemplary embodiment, the upper reboiler 13 and third separator 120 may be operated at a temperature of about −40° C. to about 0° C. and a pressure of about 1241 kPa (gauge) (180 psig) to about 3447 kPa (gauge) (500 psig).

The liquid side stream in line 134 may be heated by heat exchange in the heat exchanger 13 with the feed stream 102 to provide a heated liquid side stream in line 135. In another aspect, the liquid side stream in line 134 may be heated by heat exchange with a portion of the first vapor stream in line 112 perhaps in heat exchanger 12 to provide the heated liquid side stream in line 135. The heated liquid side stream in line 135 may be separated in a third separator 120 to provide a second vapor stream in line 122 and a second liquid stream in line 126. The second vapor stream in line 122 is passed through a second inlet 81 and fractionated in the demethanizer column 130.

A bottom reboiler stream may be produced from the bottom of the demethanizer column 130 which is heated and passed back to the bottom of the demethanizer column 130. In an aspect, the bottoms reboiler stream may be heated by heat exchange with the feed stream in line 102. In an exemplary embodiment, the demethanizer column 130 comprises two reboilers, the upper reboiler 13 and a lower reboiler 14. A lower reboiling stream may be taken from the bottom of the demethanizer column 130 in line 138. In an embodiment, the lower reboiling stream in line 138 may be taken from a third outlet 71 for the lower reboiler 14 of the demethanizer column 130. The lower reboiling stream in line 138 may be heated in the lower reboiler 14 which may comprise a heat exchanger. In an aspect, a portion of the feed stream in line 102 may be taken in line 141 and passed to the lower reboiler 14 to heat the lower reboiling stream. In this embodiment, the lower reboiling stream in line 138 may be heated by heat exchange with the feed stream in line 102 in the lower reboiler 14.

The temperature and pressure of the lower reboiler 14 controls the methane fraction dissolved in the demethanizer bottoms product in line 136. In an exemplary embodiment, the lower reboiler 14 may be operated at a temperature of about −20° C. to about 60° C. and a pressure of about 1241 kPa (gauge) (180 psig) to about 3447 kPa (gaugc) (500 psig). A heated lower reboiling stream may be taken in line 142 from the lower reboiler 14 and passed back to the bottom of the demethanizer column 130. In an embodiment, the heated lower reboiling stream in line 142 may be passed to a lower reboiling inlet 72 of the demethanizer column 130. A cooled portion of the feed stream is taken in line 143 and passed to the first separator 110.

In an aspect, the heat exchanger 13 is the upper reboiler of the demethanizer column 130. The liquid side stream in line 134 is taken as an upper reboiling stream and a heated upper reboiling stream is taken in line 135. The heated liquid side stream in line 135 is separated in the third separator 120 to provide the second vapor stream in line 122 which is recycled back to the demethanizer column 130 though the fourth inlet 81. The bottom stream from the third separator 120 in line 126 will be rich in ethane and may be taken as an ethane product stream from the demethanizer column 130. In an exemplary embodiment, the third separator 120 may be operated at a pressure of about 1241 kPa (gauge) (180 psig) to about 3447 kPa (gauge) (500 psig).

A bottoms stream comprising C2+ hydrocarbons may be produced in line 136 from the demethanizer column 130. The bottoms stream in line 136 may be taken for further recovery of ethane in the decthanizer column 160. The demethanizer column 130 may be operated at an overhead pressure of about 1241 kPa (gauge) (180 psig) to about 2068 kPa (gauge) (300 psig), and a bottoms temperature of about −30° C. (−86° F.) to about 40° C. (104° F.).

The ethane rich product stream in line 126 and demethanizer bottoms product stream in line 136 may be sent to the decthanizer column 160 downstream at different feed locations. The ethane rich product stream in line 126 has a higher ethane fraction and is colder than the demethanizer bottoms product stream in line 136. Additional heat can be recovered from the demethanizer bottoms product stream in line 136 before feeding it to the decthanizer column 160. Additional heat can also be recovered from the ethane rich product stream in line 126 before feeding it to the deethanizer.

The second liquid stream in line 126 is an ethane rich stream. The second liquid stream in line 126 may be taken for ethane recovery. In an embodiment, the second liquid stream in line 126 may be passed to a first inlet 83 of the deethanizer column 160 for separating ethane from C2+ hydrocarbons. In another embodiment, the bottoms stream in line 136 from the demethanizer column 130 may be passed as a second feed to the deethanizer column 160 via a second inlet 84. The first inlet 83 for the second liquid stream in line 126 is located above the second inlet 84 for the bottoms stream in line 136 from the demethanizer column 130. Because the second liquid stream in line 126 is ethane rich, less of the heavier material must be rectified from it.

The decthanizer column 160 produces a decthanizer overhead stream rich in ethane in the overhead line 162. The deethanizer overhead stream may be condensed in the overhead condenser 17 and separated in an overhead receiver 170. An ethane product stream may be taken in the overhead line 172 from the overhead receiver 170. An overhead liquid stream may be taken in line 174 from the overhead receiver 170 and passed to the decthanizer column 160 as reflux stream.

The decthanizer column 160 may be operated at an overhead pressure of about 1241 kPa (gauge) (180 psig) to about 2758 kPa (gauge) (400 psig), and a bottoms temperature of about 40° C. (104° F.) to about 120° C. (248° F.). A decthanized bottoms stream comprising propane may be taken in a bottoms line 164 from the decthanizer column 160.

A decthanizer reboiler stream may be taken in line 165 from the decthanized bottoms stream in line 163. The decthanizer reboiler stream in line 165 may be heated by heat exchange in a deethanizer reboiler 167 to provide a deethanized reboiled stream in line 169. The heated reboiled stream in line 169 is passed back to the bottoms of the deethanizer column 160. A net deethanized bottoms stream rich in propane is taken in line 164 from the deethanized bottoms stream in bottoms line 163.

Feeding the two streams, the second liquid stream in line 126 which is rich in ethane and the demethanizer bottoms product stream in line 136 which is leaner in ethane at different temperatures to the deethanizer column 160 at different feed inlet elevations allows for recovery of an ethane product stream in line 172 from the overhead of the deethanizer column 160 with reduced condenser 17 duty and reduced reboiler 167 duty in the decthanizer column 160.

The three demethanizer column sections 130a, 130b, and 130c can use structured packing or trays for separating products. Column internals such as accumulator trays collect vapor for the side stream 133 and collect liquid for the upper and lower reboiler draw streams. The primary trays or internals 145 and 147 are shown in the FIGURE for case of describing the working of the process.

The bottom section 130c of the demethanizer column 130 is enriched in C3+ hydrocarbons. By taking the upper reboiler draw in the second bottoms reboiler stream in line 134 from the first outlet 41 above the second liquid feed to the third inlet 61, an ethane rich liquid product in line 126 can be recovered from the third separator 120 without backmixing the ethane rich liquid in the demethanizer column 130 with the C3+ hydrocarbon rich second feed in line 121 to the third inlet 61, thereby reducing temperature at which heat is recovered in the upper reboiler 13 which is advantageous for cryogenic heat recovery. The lack of backmixing also lowers utilities for separating the ethane rich liquid in line 126 and demethanizer bottoms product stream in line 136 in a downstream decthanizer column 160.

In another aspect of the present disclosure, an apparatus for separating hydrocarbons is also disclosed. The apparatus 101 is shown in FIG. 1. The apparatus 101 comprises a first distillation column 130. The first distillation column 130 comprises a first inlet 21, and a liquid outlet 41 located below the first inlet 21 of the first distillation column 130. The liquid outlet 41 is in a downstream fluid communication with a total liquid accumulation tray of the first distillation column 130. A fourth inlet 31 is also provided in the first distillation column 130 for passing a liquid stream to the first distillation column 130 below the first inlet 21. The first distillation column 130 also comprises a third inlet 61 located below the first liquid outlet 41 in the first distillation column 130. The third inlet 61 is in downstream fluid communication with the first liquid outlet 41 of the first distillation column 130. The first distillation column 130 comprises a tray 147. A vapor outlet 51 is located below the tray 147 for taking a vapor side stream 133 from the first distillation column 130. The first distillation column 130 also comprises another tray 145 for taking a liquid side stream 134 from the first distillation column 130. In an exemplary embodiment, the first distillation column 130 comprises a total liquid accumulation tray 145 to collect all the liquid coming from above the tray.

In an embodiment, the apparatus 101 further comprises a second distillation column 160 in downstream fluid communication with the liquid outlet 41 of the first distillation column 130. In a further embodiment, the second distillation column 160 may be in downstream fluid communication with a bottoms stream 136 of the first distillation column 130.

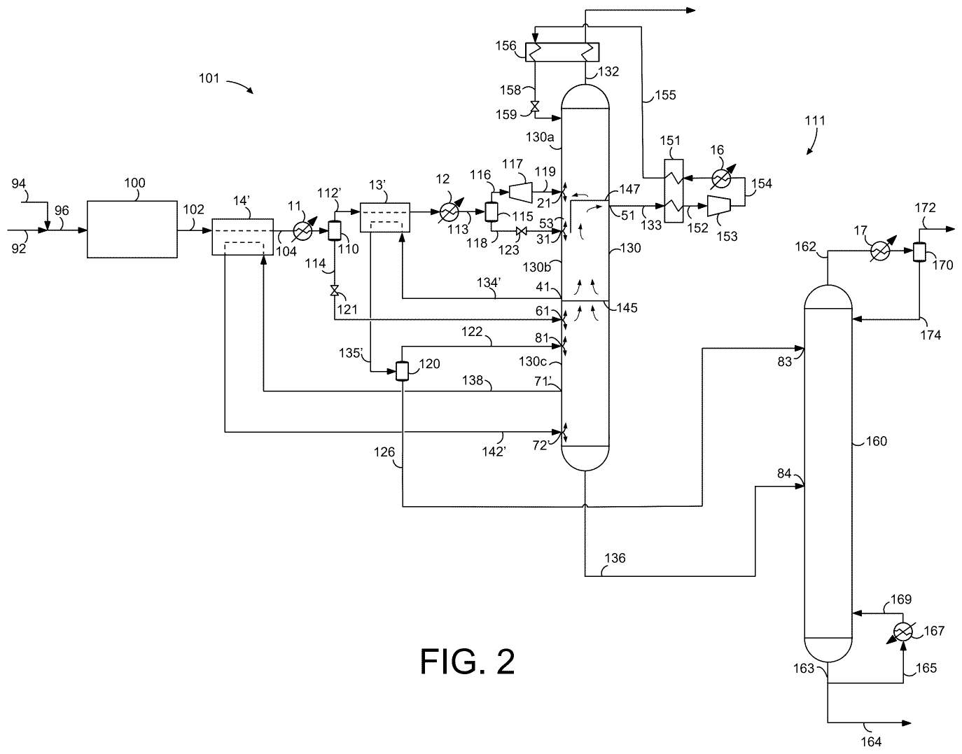

FIG. 2 shows an alternative embodiment to the embodiment of FIG. 1. Elements in FIG. 2 with the same configuration as in FIG. 1 will have the same reference numeral as in FIG. 1. Elements in FIG. 2 which have a different configuration as the corresponding element in FIG. 1 will have the same reference numeral but designated with a prime symbol (′). The configuration and operation of the embodiment of FIG. 2 is essentially the same as in FIG. 1 with the following exceptions.

In this embodiment, the liquid side stream in line 134′ is heat exchanged with the first vapor stream in line 112′ in the upper reboiler 13′. A heated liquid side stream in line 135′ is taken from the upper reboiler 13′ and passed to the third separator 120.

The lower reboiling stream may be taken from the bottom of the demethanizer column 130 in line 138 from a third outlet 71′ and heat exchanged with the feed stream in line 102 in the lower reboiler 14′. A heated lower reboiling stream in line 142′ is taken from the lower reboiler 14′ and passed to the fifth inlet 72′ of the demethanizer column 130. Rest of the process is same is described in FIG. 1.

### EXAMPLES

### Example 1

The typical separation process was simulated using process modeling software. In the typical process for Example 1, the third inlet 61 was located above the first outlet 41 of the demethanizer column 130. So, the first liquid stream in line 114 was fed to the demethanizer column 130 from the third inlet 61 at a location above the first outlet 41 for the liquid side stream in line 134. Also, the third inlet 61 and the first outlet 41 were both located above the liquid tray 145 of the demethanizer column 130. Further, the heated liquid stream in line 135 was fed directly to the demethanizer column 130 from the second inlet 81. The lower reboiling stream was taken from the bottom of the demethanizer column 130 in line 138 from the third outlet 71′ and heat exchanged with the feed stream in line 102 in the lower reboiler 14′ as shown in FIG. 2.

Feed gas stream comprising hydrogen, methane, ethane, propane, and butane in line 102 was fed to the separation unit 111 at 41° C. and 3,550 kPa(a). The feed gas was cooled by heat exchange with a demethanizer bottom stream in line 136, a lower reboiling stream in line 138, a demethanizer overhead vapor stream in line 132, an ethane product stream in line 172, and external refrigerant at −20° C. The cooled feed stream in line 104 was passed to the first separator 110 at 2° C. and 3,410 kPa(a) where a first vapor stream in line 112 was separated from a first liquid stream in line 114. The first liquid stream in line 114 was passed to the demethanizer column 130 at −6° C. and 1,750 kPa(a) at the third inlet 61.

The first vapor stream in line 112 was cooled by heat exchange with a demethanizer upper reboiling stream in line 134, the demethanizer overhead stream in line 132, and external refrigerant at −80° C. The cooled first vapor stream in line 113 was passed to the second separator 115 at −74° C. and 3,280 kPa(a) where the third vapor stream in line 116 was separated from the third liquid stream in line 118. The third liquid stream in line 118 was passed to the demethanizer column 130 at −75° C. and 1,730 kPa(a) at the fourth inlet 31. The third vapor stream in line 116 was expanded across a turboexpander 117 and passed to the demethanizer column 130 at −94° C. and 1,730 kPa(a) at the third inlet 21.

A vapor side stream in line 133 was taken from the first outlet 51 located below the first inlet 21 at −69° C. and 1,730 kPa(a). The vapor side stream in line 133 was heated to 37° C. in the side heat exchanger 151, compressed to 3,825 kPa(a) in a reflux compressor 153, cooled to 40° C. in heat exchanger 16, then cooled to −54° C. in the side heat exchanger 151. The cooled vapor side stream in line 155 was further cooled in the overhead heat exchanger 156 to −107° C. by heat exchange with the demethanizer overhead stream in line 132 at −116° C., condensing 95% of the vapor to form the reflux stream in line 158 which was expanded to −115° C. and 1,720 kPa(a) via the valve 159 and passed to the top of the demethanizer column 130. The demethanizer overhead stream in line 132 was taken from the top of the demethanizer column 130 at −116° C. and heat exchanged against the cooled vapor side stream in line 155 to −59° C., then heat exchanged against the first vapor stream to −1° C., then heat exchanged against the feed gas in line 102 to 35° C.

The demethanizer upper reboiling stream in line 134 was taken from a first outlet 41 located below the first inlet 21 at −15° C. and 1,750 kPa(a), heated to −9° C. in a demethanizer upper reboiler 13, and passed to the demethanizer column 130. The demethanizer lower reboiling stream in line 138 was taken from the third outlet 71 at −4° C. and 1,760 kPa(a), heated to 2° C. in the demethanizer lower reboiler 14′, and passed to the demethanizer column 130. The demethanizer bottoms stream in line 136 was taken from the bottom of the demethanizer column at 2° C. and 1,760 kPa(a), pumped to 1,990 kPa(a), and heated to 18° C. by heat exchange with the feed stream in line 102.

The heated demethanizer bottom stream was passed to the deethanizer column 160 where it was fractionated to form an ethane product stream in line 172 and the deethanized bottom stream in line 164.

The demethanizer column 130 achieved 99.3% ethane recovery in the demethanizer bottom stream in line 136 and 92.7% methane recovery and 100% hydrogen recovery in the demethanizer overhead stream in line 132. The deethanizer column achieves 99% ethane recovery and 95 mol % ethane purity in the ethane product stream in line 172. A summary of energy consumption for the process is provided in Table 1 below.

TABLE 1

| | | |
|---|---|---|
| Feed Refrigerant (−20° C.) | 4.5 | GJ/h |
| First Vapor Refrigerant (−80° C.) | 7.9 | GJ/h |
| Reflux Compressor 153 | 183 | kW |
| Deethanizer Condenser 17 | 11.7 | GJ/h |
| (−20° C.) | | |
| Deethanizer Reboiler 167 (LPS) | 14.7 | GJ/h |

### Example 2

The exemplary embodiment of the light paraffin separation process as shown in FIG. 2 of the present disclosure was simulated using process modeling software. The results were compared with the results of the typical separation process of Example 1.

The feed gas composition and conditions are the same as those in Example 1. Feed gas stream in line 102 enters separation unit 111 at 41° C. and 3,550 kPa(a). The feed gas stream in line 102 was cooled by heat exchange with lower reboiler stream 138 in lower reboiler 14′ at 2° C., and it was cooled in the heat exchanger 11 by heat exchange with ethane product stream in line 172 at −29° C., a demethanizer overhead stream in line 132 at −18° C., a demethanizer bottoms stream in line 136 at 12° C., and external refrigerant at −20° C. Note that exchanger 11 is representative of either a multitude of individual heat exchangers or a single multi-pass heat exchanger, or any combination thereof, and it may be placed in parallel with the lower reboiler 14′. The cooled feed stream in line 104 was passed to the first separator 110 at −14° C. and 3,410 kPa(a) where the first vapor stream in line 112′ was separated from the first liquid stream in line 114. The first liquid stream in line 114 was expanded across valve 121 to the column operating pressure 1,750 kPa(a) at −19° C. and passed to the first distillation column 130 which was a demethanizer column at the third inlet 61 located below the first outlet 41.

The first vapor stream in line 112′ was cooled by heat exchange with the liquid side stream in line 134′ in upper reboiler 13′ at −30° C., and it was cooled by heat exchange in the heat exchanger 12 by heat exchange with the demethanizer overhead stream in line 132 at −55° C. and external refrigerant at −80° C. Note that exchanger 12 is representative of either a multitude of individual heat exchangers or a single multi-pass heat exchanger, or any combination thereof, and it may be placed in parallel with the upper reboiler 13′. The cooled first vapor stream in line 113 was passed to the second separator 115 at −75° C. and 3,280 kPa(a) where the third vapor stream in line 116 was separated from the third liquid stream in line 118. The third vapor stream in line 116 was expanded across turboexpander 117 to the column operating pressure 1,730 kPa(a) at −94° C. and passed to the demethanizer column 130 at the first inlet 21. The third liquid stream in line 118 was expanded across valve 123 to the column operating pressure 1,730 kPa(a) at −76° C. and passed to the demethanizer column 130 at the fourth inlet 31.

The vapor side stream in line 133 was taken from the second outlet 51 below the first inlet 21 at −67° C. and 1,730 kPa(a). The vapor side stream in line 133 was heated to 37° C. in the side heat exchanger 151, compressed to 3,825 kPa(a) in the compressor 153, cooled to 40° C. in the heat exchanger 16, then cooled to −52° C. in the side heat exchanger 151. The cooled vapor side stream in line 155 was further cooled in the overhead heat exchanger 156 to −112° C. by heat exchange with column overhead stream in line 132 at −117° C., condensing 93% of the vapor to form the reflux stream in line 158. The reflux stream was expanded across reflux valve 159 to −118° C. at the column pressure 1,720 kPa(a) and passed to the top of the demethanizer column 130 to provide reflux for the column. The demethanizer overhead stream in line 132 was passed counter-currently to the reflux stream in heat exchanger 156 where it was heated to −55° C., counter-currently to the first vapor stream in line 112′ in the exchanger 12 where it was heated to −18° C., and counter-currently to the feed stream in line 104 in exchanger 11 where it was heated to 35° C.

The liquid side stream in line 134′ was taken from the first outlet 41 above the third inlet 61 at −30° C. and 1,750 kPa(a). The liquid side stream in line 134′ was passed counter-currently to the first vapor stream in line 112′ in upper reboiler 13′ where it was heated to −18° C. and passed to the third separator 120 forming the second vapor stream in line 122 and an ethane-rich second liquid product stream in line 126. The second vapor stream in line 122 was passed to the demethanizer column 130 at column pressure 1,750 kPa(a) at second inlet 81 located below first outlet 41. The ethane rich liquid product stream in line 172 was passed to the second distillation column 160 which was a deethanizer column at −18° C. and 1,730 kPa(a) at the first inlet 83 located near the top of the column. A lower reboiling stream 138 was taken from third outlet 71′ on the bottom stage of the column at 2° C. and 1,760 kPa(a). The lower reboiling stream was passed counter-currently to the feed stream in line 102 in lower reboiler 14′ where it was heated to 11° C. and passed to the demethanizer column 130 at lower reboiling inlet 72′.

The demethanizer bottoms product stream in line 136 was taken from the bottom of the column at 11° C. and 1,760 kPa(a). The demethanizer bottoms product stream in line 136 was pumped to 2,000 kPa(a) passed counter-currently to the feed stream in line 104 in exchanger 11 where it was heated to 26° C. The pumped, heated demethanizer bottoms product stream was then passed to the deethanizer column 160 at second inlet 84 located below the first inlet 83 near the center of the column. The deethanizer column 160 was equipped with an overhead condenser 17 utilizing refrigerant at −20° C. and a reboiler 167 utilizing low-pressure steam and fractionated the deethanizer bottoms stream in line 136 into the ethane product stream in line 172 and the deethanized bottoms stream in line 164.

The separation process of FIG. 2 achieved the same separation as that of Example 1. The demethanizer column 130 achieved 99.3% ethane recovery in the combined demethanizer bottoms product and ethane rich liquid product and 92.7% methane recovery and 100% hydrogen recovery in the demethanizer overhead stream. The deethanizer column 160 achieved 99% ethane recovery and 95 mol % ethane purity in the ethane product. A summary of energy consumption for example 2 is provided in table 2 below.

TABLE 2

| | | |
|---|---|---|
| Exchanger 11 (−20° C.) | 6.0 | GJ/h |
| Exchanger 12 (−80° C.) | 4.4 | GJ/h |
| Reflux Compressor 153 | 185 | kW |
| Deethanizer Condenser 17 | 7.3 | GJ/h |
| (−20° C.) | | |
| Deethanizer Reboiler 167 (LPS) | 12.4 | GJ/h |

Compared to Example 1, the process as shown in FIG. 2 maintains essentially the same hydrogen rejection, methane rejection, and ethane recovery in the demethanizer column when considering the combined ethane rich liquid product and demethanizer bottoms product. The process as shown in FIG. 2 had a similar reflux compressor duty around 183 to 185 kW. However, the process as shown in FIG. 2 eliminates mixing of cold, purified liquid falling down the column with warmer, heavier material contained in the first liquid stream in line 114 by drawing the liquid side stream in line 134′ from first outlet 41 above the third inlet 61 and recovering an ethane rich liquid product in line 126. The first benefit is that the liquid side stream in line 134′ in the present process at −30° C. is colder than the demethanizer upper reboiler stream of Example 1 at −15° C. which enables heat recovery at lower temperature in the upper reboiler 13′. Comparing feed refrigerant and first vapor refrigerant net utilities with exchangers 11 and 12 net utilities in Tables 1 and 2, this reduces the low temperature (−80° C.) external refrigerant duty in exchanger 12 for the present process by 3.5 GJ/h versus Example 1 at the expense of 1.5 GJ/h higher high temperature (−20° C.) external refrigerant duty in exchanger 11. The second benefit is that the present process reduces condenser and reboiler duty in the downstream deethanizer column 160 for the same ethane recovery and purity. Comparing duties in Tables 1 and 2, the process of Example 2 requires 4.4 GJ/h less high temperature (−20° C.) external refrigerant duty in the deethanizer condenser and 2.3 GJ/h less low-pressure steam (LPS) in the deethanizer reboiler than Example 1. This is a result of avoiding entropy loss by withdrawing two products from the demethanizer column 130 and passing them at optimal locations to the deethanizer column 160 compared to producing a single demethanizer bottoms product in line 136 and passing it to a single location on the deethanizer column in Example 1. The overall benefit from recovering heat at lower temperature in the upper reboiler 13′ and reducing deethanizer duty by feeding it two demethanizer products at optimal locations is that the process of Example 2 requires 18% lower high temperature external refrigerant duty at −20° C., 44% lower low temperature external refrigerant duty at −80° C., and 16% lower low-pressure steam than the process of Example 1 for the same separation in the combined demethanizer and deethanizer system.

### Specific Embodiments

While the following is described in conjunction with specific embodiments, it will be understood that this description is intended to illustrate and not limit the scope of the preceding description and the appended claims.

A first embodiment of the present disclosure is a process for separating paraffins, comprising separating a feed stream into a first vapor stream and a first liquid stream; feeding the first vapor stream to a first distillation column at a first inlet; taking a liquid side stream from the first distillation column at a first outlet; and feeding the first liquid stream to the first distillation column at a third inlet that is below the first outlet. An embodiment of the present disclosure is one, any or all of prior embodiments in this paragraph up through the first embodiment in this paragraph further comprising separating the liquid side stream into a second vapor stream and a second liquid stream; and feeding the second vapor stream to the first distillation column at a second inlet; An embodiment of the present disclosure is one, any or all of prior embodiments in this paragraph up through the first embodiment in this paragraph, wherein the liquid side stream is taken from an outlet below the first inlet of the first distillation column. An embodiment of the present disclosure is one, any or all of prior embodiments in this paragraph up through the first embodiment in this paragraph further comprising fractionating the second liquid stream in a second distillation column to separate a C2 stream from a C3+ hydrocarbon stream. An embodiment of the present disclosure is one, any or all of prior embodiments in this paragraph up through the first embodiment in this paragraph further comprising fractionating a methane stream from a C2+ bottoms stream in the first distillation column. An embodiment of the present disclosure is one, any or all of prior embodiments in this paragraph up through the first embodiment in this paragraph further comprising feeding the second liquid stream to a second distillation column at a first inlet; feeding the C2+ bottoms stream to the second distillation column at a second inlet that is below the first inlet; and fractionating an ethane stream from a C3+ stream in the second distillation column. An embodiment of the present disclosure is one, any or all of prior embodiments in this paragraph up through the first embodiment in this paragraph further comprising cooling the feed stream to provide a cooled stream; and separating the cooled stream into the first vapor stream and the first liquid stream. An embodiment of the present disclosure is one, any or all of prior embodiments in this paragraph up through the first embodiment in this paragraph wherein the feed stream comprises methane, C2+ hydrocarbons, and a non-condensable fraction. An embodiment of the present disclosure is one, any or all of prior embodiments in this paragraph up through the first embodiment in this paragraph further comprising cooling the first vapor stream to provide a cooled first vapor stream; separating the cooled first vapor stream into a third vapor stream and a third liquid stream; passing the third vapor stream to the first distillation column; and passing the third liquid stream to the first distillation column. An embodiment of the present disclosure is one, any or all of prior embodiments in this paragraph up through the first embodiment in this paragraph further comprising expanding the third vapor stream to provide an expanded third vapor stream; and passing the expanded third vapor stream to the first distillation column at the first inlet. An embodiment of the present disclosure is one, any or all of prior embodiments in this paragraph up through the first embodiment in this paragraph further comprising taking a vapor side stream from the first distillation column at a second outlet below the first inlet; cooling the vapor side stream sufficiently to condense at least a portion of the vapor side stream; and passing at least a portion of a cooled vapor side stream to the first distillation column. An embodiment of the present disclosure is one, any or all of prior embodiments in this paragraph up through the first embodiment in this paragraph further comprising taking a bottoms reboiler stream from a bottoms stream of the first distillation column; heating the bottoms reboiler stream by heat exchange with the feed stream to provide a reboiling stream and the cooled stream; passing the reboiling stream to a bottom of the first distillation column. An embodiment of the present disclosure is one, any or all of prior embodiments in this paragraph up through the first embodiment in this paragraph further comprising heating the liquid side stream by heat exchange with the feed stream to provide a heated liquid side stream; and separating the heated liquid side stream into the second vapor stream and the second liquid stream. An embodiment of the present disclosure is one, any or all of prior embodiments in this paragraph up through the first embodiment in this paragraph further comprising heating the liquid side stream by heat exchange with the first vapor stream to provide a heated liquid side stream; and separating the heated liquid side stream into the second vapor stream and the second liquid stream.

A second embodiment of the present disclosure is a process for separating paraffins, comprising separating a gaseous stream comprising methane, C2 hydrocarbons, and C3+ hydrocarbons into a first vapor stream and a first liquid stream; feeding the first vapor stream to a first distillation column at a first inlet; taking a liquid side stream from the first distillation column at a first outlet; separating the liquid side stream into a second vapor stream and a second liquid stream; feeding the second vapor stream to the first distillation column at a second inlet; feeding the first liquid stream to the first distillation column at a third inlet that is below the first outlet; and fractionating the second liquid stream in a second distillation column to separate a C2 hydrocarbon stream from a C3+ hydrocarbon stream. An embodiment of the present disclosure is one, any or all of prior embodiments in this paragraph up through the second embodiment in this paragraph, wherein the liquid stream is taken from an outlet of the first distillation column below an inlet for the first vapor stream. An embodiment of the present disclosure is one, any or all of prior embodiments in this paragraph up through the second embodiment in this paragraph further comprising cooling the first vapor stream to provide a cooled first vapor stream; separating the cooled first vapor stream into a third vapor stream and a third liquid stream; passing the third vapor stream to the first distillation column at a first inlet; and passing the third liquid stream to the first distillation column at a fourth inlet at or below the first inlet. An embodiment of the present disclosure is one, any or all of prior embodiments in this paragraph up through the second embodiment in this paragraph further comprising fractionating a methane stream from a C2+ bottoms stream in the first distillation column; feeding the second liquid stream to the second distillation column at a first inlet; and feeding the C2+ bottoms stream to the second distillation column at a second inlet that is below the first inlet. An embodiment of the present disclosure is one, any or all of prior embodiments in this paragraph up through the second embodiment in this paragraph further comprising heating the liquid side stream by heat exchange with the first vapor stream to provide a heated liquid side stream; and separating the heated liquid side stream into the second vapor stream and the second liquid stream.

A third embodiment of the present disclosure is an apparatus for separating hydrocarbons, comprising a first distillation column comprising a first inlet; a liquid outlet located below the first inlet of the distillation column and in fluid communication with a total liquid accumulation tray; and a second inlet located below the liquid outlet in the distillation column. An embodiment of the present disclosure is one, any or all of prior embodiments in this paragraph up through the third embodiment in this paragraph further comprising a second distillation column in fluid communication with the liquid outlet of the first distillation column. An embodiment of the present disclosure is one, any or all of prior embodiments in this paragraph up through the third embodiment in this paragraph further comprising an upper reboiler and a lower reboiler.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for separating paraffins, comprising:
separating a feed stream into a first vapor stream and a first liquid stream;
feeding said first vapor stream to a first distillation column at a first inlet;
taking a liquid side stream from the first distillation column at a first outlet; and
feeding said first liquid stream to the first distillation column at a third inlet that is below the first outlet.

2. The process of claim 1 further comprising:
separating said liquid side stream into a second vapor stream and a second liquid stream; and
feeding said second vapor stream to the first distillation column at a second inlet.

3. The process of claim 1, wherein said liquid side stream is taken from an outlet below said first inlet of the first distillation column.

4. The process of claim 2 further comprising fractionating said second liquid stream in a second distillation column to separate a C2 stream from a C3+ hydrocarbon stream.

5. The process of claim 2 further comprising:
fractionating a methane stream from a C2+ bottoms stream in said first distillation column.

6. The process of claim 5 further comprising:
feeding said second liquid stream to a second distillation column at a first inlet;
feeding said C2+ bottoms stream to the second distillation column at a second inlet that is below the first inlet; and
fractionating an ethane stream from a C3+ stream in the second distillation column.

7. The process of claim 1 further comprising:
cooling said feed stream to provide a cooled stream; and
separating said cooled stream into said first vapor stream and said first liquid stream.

8. The process of claim 1 wherein said feed stream comprises methane, C2+ hydrocarbons, and a non-condensable fraction.

9. The process of claim 1 further comprising:
cooling said first vapor stream to provide a cooled first vapor stream;
separating said cooled first vapor stream into a third vapor stream and a third liquid stream;
passing said third vapor stream to the first distillation column at the first inlet; and
passing said third liquid stream to the first distillation column.

10. The process of claim 9 further comprising:
expanding said third vapor stream to provide an expanded third vapor stream; and
passing said expanded third vapor stream to the first distillation column at the first inlet.

11. The process of claim 9 further comprising:
taking a vapor side stream from the first distillation column at a second outlet below the first inlet;
cooling said vapor side stream sufficiently to condense at least a portion of said vapor side stream; and
passing at least a portion of a cooled vapor side stream to the first distillation column.

12. The process of claim 1 further comprising:
taking a bottoms reboiler stream from a bottoms stream of the first distillation column;
heating said bottoms reboiler stream by heat exchange with said feed stream to provide a reboiling stream and said cooled stream; and
passing said reboiling stream to a bottom of the first distillation column.

13. The process of claim 1 further comprising:
heating said liquid side stream by heat exchange with said feed stream to provide a heated liquid side stream; and
separating said heated liquid side stream into said second vapor stream and said second liquid stream.

14. The process of claim 1 further comprising:
heating said liquid side stream by heat exchange with said first vapor stream to provide a heated liquid side stream; and
separating said heated liquid side stream into said second vapor stream and said second liquid stream.

15. A process for separating paraffins, comprising:
separating a gaseous stream comprising methane, C2 hydrocarbons, and C3+ hydrocarbons into a first vapor stream and a first liquid stream;
feeding said first vapor stream to a first distillation column at a first inlet;
taking a liquid side stream from the first distillation column at a first outlet;
separating said liquid side stream into a second vapor stream and a second liquid stream;
feeding said second vapor stream to the first distillation column at a second inlet;
feeding said first liquid stream to the first distillation column at a third inlet that is below the first outlet; and
fractionating said second liquid stream in a second distillation column to separate a C2 hydrocarbon stream from a C3+ hydrocarbon stream.

16. The process of claim 15, wherein said liquid side stream is taken from an outlet of the first distillation column below an inlet for said first vapor stream.

17. The process of claim 15 further comprising:
cooling said first vapor stream to provide a cooled first vapor stream;
separating said cooled first vapor stream into a third vapor stream and a third liquid stream;
passing said third vapor stream to the first distillation column at a first inlet; and
passing said third liquid stream to the first distillation column at a fourth inlet at or below the first inlet.

18. The process of claim 15 further comprising:
fractionating a methane stream from a C2+ bottoms stream in said first distillation column;
feeding said second liquid stream to the second distillation column at a first inlet; and
feeding said C2+ bottoms stream to the second distillation column at a second inlet that is below the first inlet.

19. The process of claim 15 further comprising:
heating said liquid side stream by heat exchange with said first vapor stream to provide a heated liquid side stream; and
separating said heated liquid side stream into said second vapor stream and said second liquid stream.

20. An apparatus for separating hydrocarbons, comprising:
a first distillation column comprising a first inlet;
a liquid outlet located below said first inlet of the distillation column and in fluid communication with a total liquid accumulation tray; and
a second inlet located below said liquid outlet in the distillation column.

21. The apparatus of claim 18 further comprising a second distillation column in fluid communication with the liquid outlet of the first distillation column.

22. The apparatus of claim 18 further comprising an upper reboiler and a lower reboiler.
